(19) United States
(12) Patent Application Publication
(10) Pub. No.: US 2026/0146223 A1
(43) Pub. Date: May 28, 2026

(54) BIOREACTOR SYSTEM

(71) Applicant: AMOGREENTECH CO., LTD., Gimpo-si (KR)

(72) Inventors: Song-Hee KOO, Gimpo-si (KR); Su-Yeon LEE, Gimpo-si (KR); Hee Sung PARK, Gimpo-si (KR); Hyeong Tak NOH, Gimpo-si (KR); Ji Young KIM, Gimpo-si (KR)

(73) Assignee: AMOGREENTECH CO., LTD., Gimpo-si (KR)

(21) Appl. No.: 19/122,758

(22) PCT Filed: Oct. 19, 2023
(86) PCT No.: PCT/KR2023/016259

§ 371 Date: Apr. 21, 2025

(30) Foreign Application Priority Data
Oct. 21, 2022 (KR) 10-2022-0136292

(51) Int. Cl. C12M 1/00; C12M 1/04; C12M 1/107; C12M 1/12; C12M 3/00

(52) U.S. Cl. CPC C12M 29/14; C12M 23/24; C12M 23/34; C12M 23/36; C12M 23/48; C12M 25/06; C12M 29/18; C12M 37/02

(57) ABSTRACT

A bioreactor system is provided, including: a reactor unit for culturing/proliferating cells or secretion of exosomes from cells; a solution supply unit in which a certain amount of a solution to be supplied to the reactor unit is stored; a circulation pump that is mutually connected to the reactor unit and the solution supply unit by means of a tube and circulates the solution stored in the solution supply unit; and a gas supply unit which includes a main body having a gas storage space filled with gas supplied from outside and is installed in the solution supply unit so as to supply the gas stored in the gas storage space to the solution stored in the solution supply unit, wherein the main body may be arranged to be submerged in the solution filled in the solution supply unit so that the gas can move toward the solution and dissolve.

### CROSS REFERENCE TO THE RELATED APPLICATIONS

This application is the national phase entry of International Application No. PCT/KR 2023/016259, filed on Oct. 19, 2023, which is based upon and claims priority to Korean Patent Application No. 10-2022-0136292, filed on Oct. 21, 2022, the entire contents of which are incorporated herein by reference.

### TECHNICAL FIELD

The present invention relates to a bioreactor system.

### BACKGROUND

A bioprocess refers to a process in the biotechnology field in which living cells are used to produce a desired therapeutic agent. Antibodies, stem cells, immune cells, and the like are produced through cell culture and used to manufacture biopharmaceuticals, vaccines, and cell therapeutic agents.

In addition, exosomes are substances that mediate intercellular signaling and are naturally secreted from cells during the process of cell proliferation/culture. Such exosomes contain various types of information from parent cells, such as proteins and DNA, and are being developed for use as biomarkers or therapeutic agents.

Meanwhile, cells are classified into adherent cells, which need to attach to a surface matrix, and suspension cells, which proliferate without attachment to the surface of a matrix.

That is, adherent cells are cultured while attached to a scaffold that serves as a surface matrix, whereas suspension cells are not cultured while attached to a scaffold but instead grow by receiving signals as they repeat a contact-suspension-contact-suspension process in which they attach to and detach from the surface of the scaffold.

A bioreactor is used to culture such cells or to obtain exosomes naturally secreted from the cells, and the internal environment in which the cells are placed within the bioreactor must be maintained under appropriate conditions so that the cells can be smoothly cultured/proliferated and can smoothly secrete exosomes.

That is, the medium that supplies nutrients during cell culture in the bioreactor must maintain a constant pH suitable for cell growth, and a larger amount of exosomes can be secreted from the cells when the conditions are similar to those under which the cells are cultured/proliferated.

To this end, a method of continuously supplying an appropriate gas to the medium or buffer solution during cell culture/proliferation is employed to promote smooth cell growth and facilitate the secretion of exosomes.

However, if air bubbles are included during the process of supplying gas to the medium or buffer solution, the cells may not be able to smoothly receive the nutrients contained in the medium due to the air bubbles, or may suffer from stress caused by the bubbles, resulting in reduced growth or impaired secretion of exosomes.

### SUMMARY

### Technical Problem

The present invention was devised in view of the above points, and is directed to providing a bioreactor system capable of smoothly supplying gas to a solution while suppressing the generation of air bubbles during gas supply.

In addition, the present invention is also directed to providing a bioreactor system capable of easily changing the oxygen concentration of the supplied gas.

### Technical Solution

In order to solve the above problems, the present invention provides a bioreactor system, including a reactor unit for culturing or proliferating cells or for secretion of exosomes from the cells; a solution supply unit in which a certain amount of a solution to be supplied to the reactor unit is stored; a circulation pump that is connected to the reactor unit and the solution supply unit via a tube and configured to circulate the solution stored in the solution supply unit; and a gas supply unit which includes a main body having a gas storage space filled with gas supplied from the outside and is installed in the solution supply unit so as to supply the gas stored in the gas storage space to the solution stored in the solution supply unit, wherein the main body is arranged to be submerged in the solution filled in the solution supply unit so that the gas stored in the gas storage space can toward the solution and dissolve.

In addition, the gas supply unit may include a main body having a gas storage space filled with gas supplied from the outside; an opening formed in the main body to communicate with the gas storage space; a plate-shaped porous member surrounding the main body to cover the opening; a gas inlet provided in the main body to allow gas to flow into the gas storage space; and a gas outlet provided in the main body to allow the gas in the gas storage space to be discharged to the outside.

In addition, the porous member may be formed of a hydrophobic membrane to allow the gas to move from the gas storage space toward the solution through the opening while preventing the solution stored in the solution supply unit from entering the gas storage space.

In addition, the bioreactor system may further include a sterilization filter connected to the gas inlet.

In addition, the gas supply unit may include a plurality of openings spaced apart along a circumferential direction of the main body, and the openings may be formed in the main body to have a predetermined area.

In addition, the bioreactor system may further include a frame unit including: a first mounting base for supporting the solution supply unit; a second mounting base disposed to be spaced apart from the first mounting base by a predetermined distance and configured to support the reactor unit; and a support plate interconnecting the first mounting base and the second mounting base and on which the circulation pump is mounted. Through this, the bioreactor system may be implemented in a portable form.

In addition, the reactor unit may include a reactor housing formed in the shape of an enclosure and having an accommodating space; a plurality of scaffolds provided in a plate shape having a predetermined area and arranged in parallel with each other at a distance, with one surface of each facing each other in the accommodating space; an inlet provided in the reactor housing to introduce the solution circulated by the circulation pump into the accommodating space; and an outlet provided in the reactor housing to discharge the medium in the accommodating space to the solution supply unit.

In addition, the reactor unit may include a plurality of scaffolds arranged in parallel with each other at a distance, with one surface of each facing each other, each of the plurality of scaffolds may include a plate-shaped nanofiber membrane coated with a protein motif; and a support member attached to one surface of the nanofiber membrane via an adhesive layer so as to support the nanofiber membrane.

In addition, the solution supply unit may include an enclosure-shaped supply housing having an internal space in which a predetermined amount of the solution is stored; a discharge port provided in the supply housing to supply the solution stored in the internal space to the reactor unit; and a return port provided in the supply housing to return the solution from the reactor unit to the internal space, and the main body may be disposed to be submerged in the solution filled in the internal space.

In this case, the internal space may include a first space and a second space that are partitioned from each other while being in communication with each other via a partition member extending a predetermined height from a floor surface of the supply housing, the return port may be provided in the supply housing to be in communication with the first space, and the discharge port may be provided in the supply housing to be in communication with the second space. In this case, the main body may be disposed to be submerged in the solution filled in the first space.

In addition, the gas may be a mixed gas having an oxygen concentration of 2% to 14%.

In addition, the solution may be a medium or a buffer solution.

### Advantageous Effect

According to the present invention, gas from which air bubbles have been removed can be supplied to a medium or buffer solution through the gas supply unit. Through this, the bioreactor of the present invention not only enables stable cell culture but also increases the amount of exosome secretion.

In addition, according to the present invention, the oxygen concentration of the gas supplied through the gas supply unit can be easily adjusted, thereby enabling cell culture or exosome production in an environment similar to that of the human body.

Furthermore, according to the present invention, the entire system can be implemented in a portable form, allowing cells to be cultured or proliferated and exosomes secreted from the cells to be obtained in various locations without spatial limitations.

### BRIEF DESCRIPTION OF THE DRAWINGS

FIG. 1 is an illustrated view of a bioreactor system according to an exemplary embodiment of the present invention.

FIG. 2 is an illustrated view of a reactor unit applicable to the bioreactor system according to an exemplary embodiment of the present invention.

FIG. 3 is an exploded view of FIG. 2.

FIG. 4 is a cross-sectional view taken along line A-A of FIG. 2.

FIG. 5 is an exploded view of a scaffold assembly shown in FIG. 3.

FIGS. 6A-6B are cross-sectional views illustrating a detailed structure of a scaffold applicable to the bioreactor system according to an exemplary embodiment of the present invention.

FIG. 7 is an illustrated view of a solution supply unit applicable to the bioreactor system according to an exemplary embodiment of the present invention.

FIG. 8 is an exploded view of FIG. 7.

FIG. 9 is a cross-sectional view taken along line B-B of FIG. 7.

FIG. 10 is an illustrated view of a gas supply unit applicable to the bioreactor system according to an exemplary embodiment of the present invention.

FIG. 11 is an illustrated view showing a porous member in a separated state relative to FIG. 10.

FIG. 12 is a cross-sectional view taken along line C-C of FIG. 10.

FIG. 13 is an illustrated view of another type of gas supply unit applicable to the bioreactor system according to an exemplary embodiment of the present invention.

### DETAILED DESCRIPTION OF THE EMBODIMENTS

Hereinafter, exemplary embodiments of the present invention will be described in detail so that those of ordinary skill in the art can readily practice the present invention with reference to the accompanying drawings. The present invention may be embodied in many different forms and is not limited to the embodiments set forth herein. In the drawings, parts unrelated to the description are omitted for clarity of description of the present invention, and the same or similar reference numerals denote the same or corresponding elements.

A bioreactor system 100 according to an exemplary embodiment of the present invention may culture/proliferate cells or obtain exosomes naturally secreted from the cells by using a solution and a plurality of scaffolds 116 and 116′, which are arranged in an accommodating space S1 of a reactor unit 110 along with a certain amount of the solution.

In the present invention, the exosome (e) may be a substance that mediates intercellular signaling and a vesicle having a size ranging from 30 nm to 200 nm or less.

The solution filled in the accommodating space (S1) may be a medium containing nutrients required for cell culture or proliferation, or a buffer solution that does not react with cells or exosomes.

In such a case, the buffer solution may be mixed with the exosomes and may serve as a solution that protects the cells or exosomes. By way of example, the buffer solution may be a known phosphate-buffered saline (PBS).

The medium may further include magnetic particles coated with a peptide motif.

Accordingly, when a certain amount of the medium is filled in the accommodating space (S1), the bioreactor system 100 according to an exemplary embodiment of the present invention may culture/proliferate cells using nutrients supplied from the medium.

In addition, when a certain amount of the buffer solution is filled in the accommodating space (S1), the bioreactor system 100 according to an exemplary embodiment of the present invention may obtain exosomes secreted from the cells attached to the scaffolds 116 and 116′.

Here, the cells may be adherent cells that are attached to the scaffolds 116 and 116′ constituting the scaffold assembly (P).

In this case, the bioreactor system 100 according to an exemplary embodiment of the present invention may be implemented as a solution circulation-type system that circulates the solution filled in the accommodating space (S1) and is also capable of supplying gas to the circulating solution.

Through this, the bioreactor system 100 according to an exemplary embodiment of the present invention may allow the circulating solution to be reused while enabling the cells to be smoothly cultured/proliferated or to secrete exosomes in the reactor unit 110.

In such a case, the gas supplied to the solution may be supplied to the solution (L) in a state in which air bubbles have been removed.

By way of example, the gas may be carbon dioxide, but is not limited thereto and may be appropriately changed depending on the type of cells, and the gas may be a mixed gas containing carbon dioxide, oxygen, and the like.

In addition, when the gas includes oxygen, the gas may be a mixed gas having an oxygen concentration of 2 to 14%.

As a result, even if the solution is reused through circulation, it may be maintained in a suitable state for cell culture or survival through the supply of the gas. By way of example, the solution may be maintained at an appropriate pH required for cell culture through gas dissolution.

In addition, when the gas supplied to the solution is a mixed gas having an oxygen concentration of 2 to 14%, the interior of the reactor unit 110 may be maintained in a hypoxic environment similar to that of the human body in the bioreactor system 100 according to an exemplary embodiment of the present invention, so that the cells or exosomes obtained through the bioreactor system 100 may be in a state similar to that of human cells or exosomes.

To this end, the bioreactor system 100 according to an exemplary embodiment of the present invention may include a reactor unit 110, a solution supply unit 120, a circulation pump 130, and gas supply units 140 and 140′.

The reactor unit 110 may culture/proliferate cells or allow exosomes to be secreted from the cells while a certain amount of the solution (L) is filled therein.

To this end, the reactor unit 110 may include a plurality of scaffolds 116 and 116′ disposed therein, and the plurality of scaffolds 116 and 116′ may enable cells to be cultured or proliferated and to secrete exosomes.

That is, the reactor unit 110 may include an accommodating space (S1) that accommodates the solution (L) and the plurality of scaffolds 116 and 116′ to which adherent cells are attached, and may include an inlet 114 for introducing the solution supplied from the solution supply unit 120 into the accommodating space (S1), and an outlet 115 for discharging the solution in the accommodating space (S1) to the outside.

In such a case, the inlet 114 of the reactor unit 110 may be connected to the circulation pump 130, which is connected to the solution supply unit 120 via a connection line 151, and the outlet 115 may be connected to the solution supply unit 120 via a connection line 152.

Accordingly, the solution that has moved from the solution supply unit 120 into the accommodating space (S1) through the inlet 114 may pass through spaces formed between the scaffolds 116 and 116′ disposed in the accommodating space (S1), and may then be returned to the solution supply unit 120 through the outlet 115.

Here, cells to be cultured or cells for secreting exosomes may be attached to the plurality of scaffolds 116 and 116′, and when the solution filled in the accommodating space (S1) is a medium, the cells attached to the scaffolds 116 and 116′ may receive nutrients from the medium.

Through this, the cells attached to the plurality of scaffolds 116 and 116′ may be smoothly cultured or proliferated through nutrients supplied from the solution, or may secrete exosomes toward the solution filled in the accommodating space (S1).

In this case, the plurality of scaffolds 116 and 116′ may be provided in a plate shape having a predetermined area, and the plurality of plate-shaped scaffolds 116 and 116′ may be arranged in parallel at intervals from each other, with one surface of each facing each other in the accommodating space (S1).

As a result, the bioreactor system 100 according to an exemplary embodiment of the present invention may increase the integration density of the scaffolds 116 and 116′ arranged in the accommodating space (S1), thereby enabling a large number of cells to be cultured or proliferated through a single culture process and increasing the total number of cells attached to the plurality of scaffolds 116 and 116′, which in turn may increase the amount of exosomes secreted from the cells.

In addition, since a plurality of scaffolds 116 and 116′ can be arranged in parallel in a single reactor unit 110, the bioreactor system 100 according to an exemplary embodiment of the present invention may reduce the overall size of the reactor unit 110.

To this end, the scaffolds 116 and 116′ may be implemented in a plate shape, and any known material may be used without limitation as long as it allows cells to be easily attached.

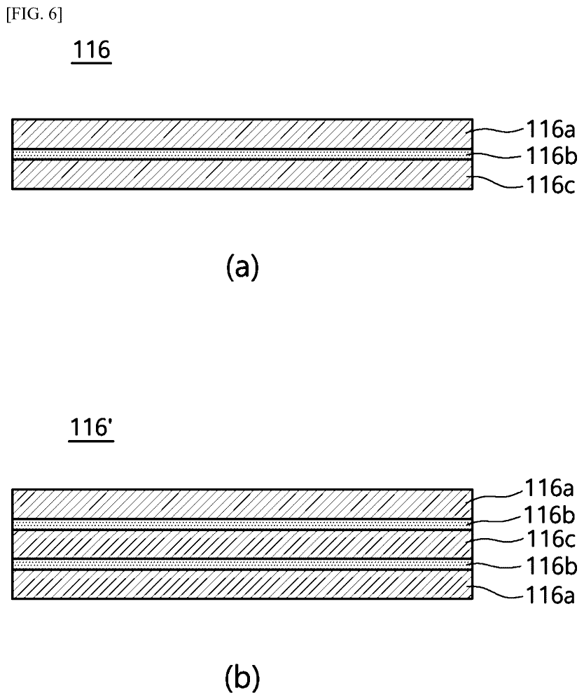

As a non-limiting example, the scaffolds 116 and 116′may include a nanofiber membrane 116a in which nanofibers are formed into a three-dimensional network structure through electrospinning. In such a case, the scaffold 116 may have a three-layer structure including the nanofiber membrane 116a, as illustrated in FIG. 6A, and a support member 116c attached to one surface of the nanofiber membrane 116a via an adhesive layer 116b.

Here, the support member 116c may be a plate-shaped film member and may support one surface of the nanofiber membrane 116a. Through this, even if the nanofiber membrane 116a is formed in a plate shape with flexibility, it can be supported by the support member 116c, thereby preventing bending or sagging. Accordingly, the scaffold 116 disposed in the accommodating space S1 of the reactor unit 110 can maintain an unfolded state.

However, the structure of the scaffold 116 is not limited thereto, and the scaffold 116′ may have a five-layer structure in which the nanofiber membrane 116a is attached to both surfaces of the support member 116c via the adhesive layer 116b, as illustrated in FIG. 6B.

Furthermore, the scaffolds 116 and 116′ may have a surface that is modified to allow cells to be smoothly attached. That is, the nanofiber membrane 116a may be a membrane in which the surfaces of the nanofibers are motif-coated. Accordingly, cells can be smoothly attached to the surfaces of the scaffolds 116 and 116′.

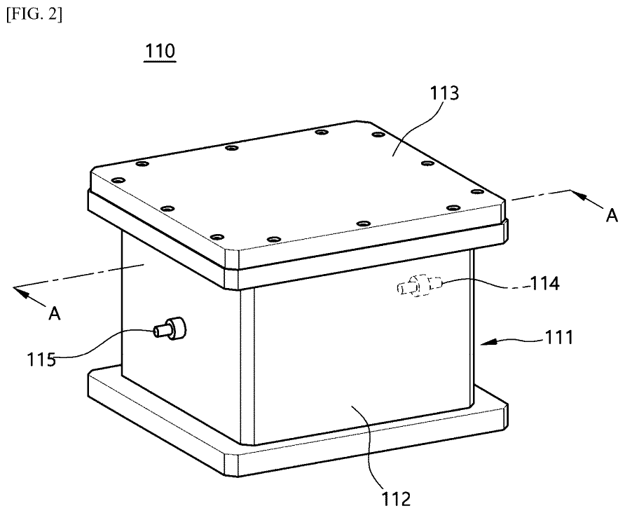

The reactor unit 110 as described above may include a reactor housing 111 and the above-described scaffolds 116 and 116′, as illustrated in FIGS. 2 to 4, and the reactor housing 111 may include an inlet 114 and an outlet 115, as described above.

Specifically, the reactor housing 111 may accommodate a certain amount of solution and a plurality of scaffolds 116 and 116′ therein, as described above.

To this end, the reactor housing 111 may be formed in the shape of an enclosure having an accommodating space S1.

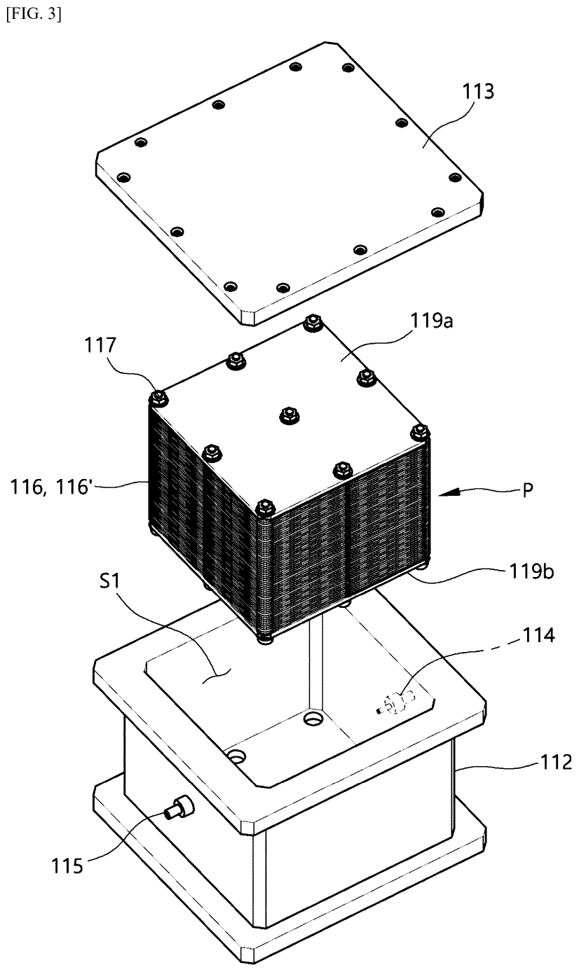

For example, as illustrated in FIG. 3, the reactor housing 111 may include a housing body 112 in the shape of an enclosure having an accommodating space S1 that is open at the top, the inlet 114 and the outlet 115 may be respectively formed on one side of the housing body 112, and the accommodating space S1 open at the top may be sealed by a cover member 113 coupled to the housing body 112.

Through this, the solution supplied from the solution supply unit 120 to the reactor unit 110 can fill the accommodating space S1 through the inlet 114, and the solution filled in the accommodating space S1 can be discharged to the outside through the outlet 115.

In this case, the plurality of scaffolds 116 and 116′ disposed in the accommodating space S1 may be configured in the form of an assembly so as to increase integration density while improving assemblability.

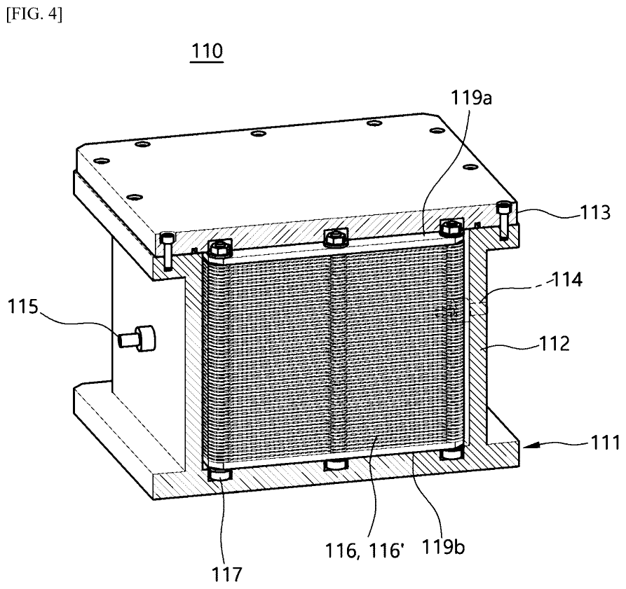

For example, as illustrated in FIGS. 3 and 4, the reactor unit 110 may include a scaffold assembly (P) inserted and disposed in the accommodating space S1, and the scaffold assembly (P) may be formed by integrating a plurality of scaffolds 116 and 116′ with a predetermined spacing therebetween.

Specifically, the scaffold assembly (P) may be configured in a stacked form in which the plurality of scaffolds 116 and 116′ are spaced apart in parallel along the height direction of the housing body 112.

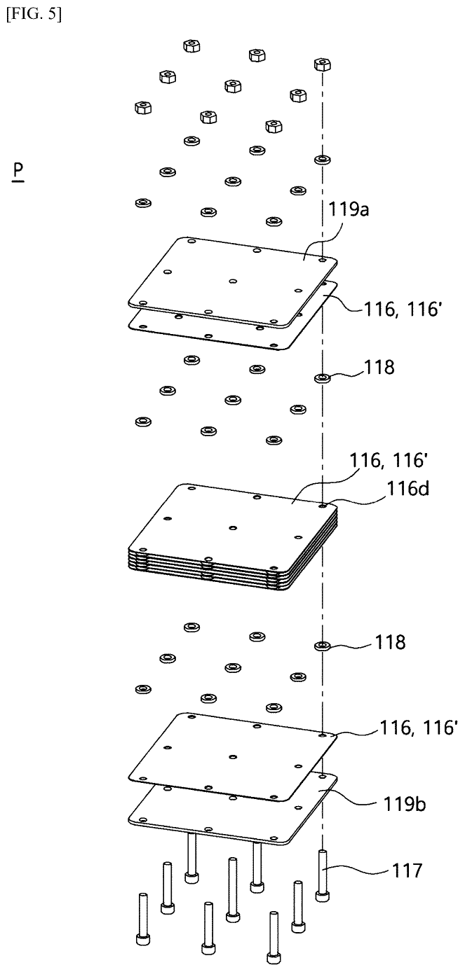

To this end, the scaffold assembly (P) may include a plurality of fastening bars 117 having a predetermined length and a plurality of spacing members 118 formed in a ring shape, as illustrated in FIG. 5, and the plurality of scaffolds 116 and 116′ may be fitted onto the fastening bars 117, respectively.

In this case, the plurality of fastening bars 117 may be spaced apart from each other at predetermined intervals, and both end portions of the plurality of fastening bars 117 may be fixed to a plate-shaped upper plate 119a and lower plate 119b, each having a predetermined area.

Accordingly, the plurality of fastening bars 117, with both ends respectively fixed to the upper plate 119a and the lower plate 119b, can remain spaced apart from each other, and the plurality of scaffolds 116 and 116′ can be arranged in parallel with each other between the upper plate 119a and the lower plate 119b. The plurality of scaffolds 116 and 116′ may be fastened to the respective fastening bars 117 through a plurality of through holes 116d formed at positions corresponding to the plurality of fastening bars 117, respectively.

In addition, the plurality of spacing members 118, like the scaffolds 116 and 116′, may be fitted onto the plurality of fastening bars 117, respectively, and the plurality of spacing members 118 and the plurality of scaffolds 116 and 116′ may be alternately fastened to each of the fastening bars 117.

Accordingly, each of the spacing members 118 may be disposed between two scaffolds 116 and 116′ arranged in parallel with each other, and the two scaffolds 116 and 116′ disposed above and below may maintain a spaced interval from each other through the spacing members 118.

However, the scaffold assembly (P) is not limited thereto, and any known method may be applied as long as the plurality of scaffolds 116 and 116′ are arranged in parallel with each other in one direction and can maintain a predetermined spaced interval therebetween.

The solution supply unit 120 may serve to supply a solution to the reactor unit 110, and as described above, the solution stored inside the solution supply unit 120 may be supplied to the reactor unit 110 through the circulation pump 130 and then returned from the reactor unit 110 back to the solution supply unit 120.

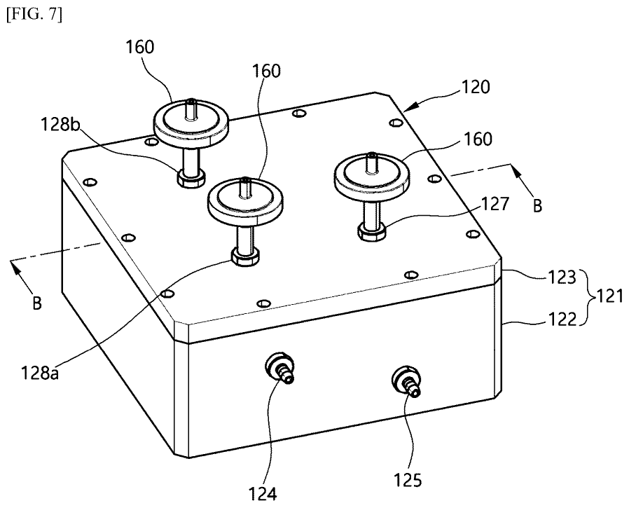

To this end, the solution supply unit 120 may include a supply housing 121 in the shape of an enclosure having an internal space (S2) for storing a predetermined amount of the solution, as illustrated in FIGS. 7 to 9. The supply housing 121 may include a discharge port 125 and a return port 124, which allow the solution stored inside the housing to be circulated by the operation of the circulation pump 130, supplied to the reactor unit 110, and then returned back to the supply housing 121.

In this case, the discharge port 125 may serve as a supply port for supplying the solution stored in the internal space (S2) to the reactor unit 110, and the return port 124 may serve as an inlet port for returning the solution from the reactor unit 110 to the internal space (S2).

Here, the internal space (S2) may be formed to be open at the top. In this case, the supply housing 121 may further include a housing main body 122 in the shape of an enclosure having the internal space (S2), and a lid member 123 that covers the open top of the internal space (S2).

In addition, as described above, the discharge port 125 may be connected to the inlet 114 of the reactor unit 110 via the connection line 151, and the return port 124 may be connected to the outlet 115 of the reactor unit 110 via the connection line 152.

Through this, the solution stored in the internal space (S2) may circulate between the accommodating space (S1) of the reactor unit 110 and the internal space (S2) of the solution supply unit 120 by operation of the circulation pump 130.

That is, the solution stored in the internal space (S2) may be transferred from the internal space (S2) to the accommodating space S1 by operation of the circulation pump 130 and then returned to the internal space (S2).

In this case, the internal space S2 may be partitioned into at least two spaces.

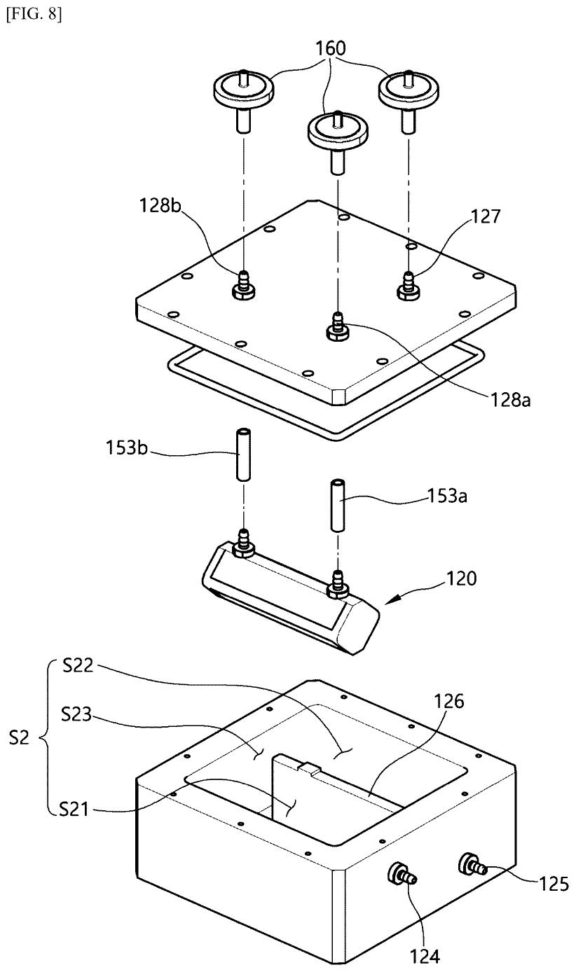

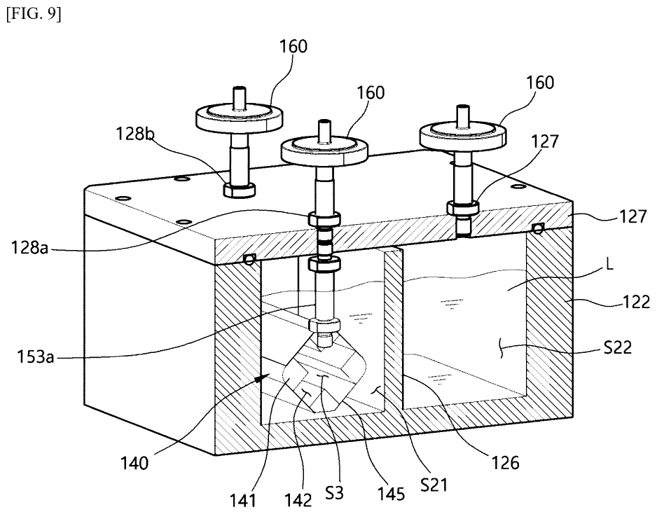

For example, as illustrated in FIGS. 8 and 9, the supply housing 121 may include a plate-shaped partition member 126 extending upward from the floor surface of the housing main body 122 that defines the internal space (S2), and the partition member 126 may divide the internal space (S2) into a first space (S21) and a second space (S22).

In this case, the partition member 126 may extend upward from the floor surface of the housing main body 122 to a predetermined height, with one end connected to an inner surface of the housing main body 122 and the other end spaced apart from another inner surface of the housing main body 122 by a predetermined distance.

Accordingly, the first space (S21) and the second space (S22) may be connected to each other through a communication passage (S23) formed at one end of the partition member 126.

In this case, the return port 124 may be provided in the housing main body 122 so as to be in communication with the first space (S21), and the discharge port 125 may be provided in the housing main body 122 so as to be in communication with the second space (S22). The return port 124 and the discharge port 125 may be provided in the housing main body 122 such that they are positioned on the same surface thereof.

Through this, the solution returned to the internal space (S2) through the return port 124 after being transferred from the accommodating space (S1) of the reactor unit 110 may pass through the first space (S21), the communication passage (S23), and the second space (S22), and then be transferred toward the accommodating space (S1) through the discharge port 125.

Accordingly, the solution transferred from the return port 124 to the discharge port 125 may have an increased total travel distance within the internal space (S2).

As a result, even if the solution returned to the internal space (S2) contains air bubbles, the air bubbles included in the solution may float upward due to buoyancy during the movement of the solution from the return port 124 to the discharge port 125 within the internal space (S2) and may be completely separated from the solution.

Accordingly, the solution supplied to the accommodating space (S1) of the reactor unit 110 through the discharge port 125 may be maintained in an optimal state with impurities such as air bubbles removed.

In addition, the return port 124 may be provided in the housing main body 122 so as to be positioned at a relatively higher location than the discharge port 125. That is, the discharge port 125 may be provided so as to be positioned relatively closer to the floor surface of the housing main body 122, which defines the bottom of the internal space (S2), than the return port 124.

Through this, the medium introduced into the first space (S21) from the reactor unit 110 through the return port 124 may be supplied back to the reactor unit 110 through the discharge port 125, which is formed at a position relatively lower than the return port 124.

As a result, even if the solution recovered to the first space (S21) through the return port 124 contains air bubbles, the air bubbles contained in the solution can be completely separated from the solution by moving upward due to buoyancy during the process in which the solution returned to the internal space (S2) moves from the return port 124 toward the discharge port 125, which is formed at a position relatively lower than the return port 124.

Accordingly, the solution supplied to the accommodating space (S1) of the reactor unit 110 through the discharge port 125 may be maintained in an optimal state with impurities such as air bubbles removed.

As such, the bioreactor system 100 according to an exemplary embodiment of the present invention may simply implement a closed circulation system by interconnecting the reactor unit 110, the circulation pump 130, and the solution supply unit 120 such that the solution stored in the solution supply unit 120 circulates through the reactor unit 110 and the solution supply unit 120 by the operation of the circulation pump 130.

In this case, the solution supply unit 120 may further include a vent port 127 provided in the supply housing 121. The vent port 127 may be formed in the supply housing 121 so as to be in communication with the internal space (S2).

The vent port 127 may serve to discharge air present in the internal space (S2) to the outside. For example, as described above, the vent port 127 may discharge air bubbles separated from the solution in the internal space (S2) to the outside.

The gas supply unit 140, 140′ may be installed on the solution supply unit 120 and may be disposed in the internal space (S2) to supply gas for cell culture/proliferation or exosome secretion to the solution circulating through the reactor unit 110 and the solution supply unit 120.

Through this, even when the solution filled in the internal space S2 is reused while circulating through the reactor unit 110 and the solution supply unit 120 in the bioreactor system 100 according to an exemplary embodiment of the present invention, the solution returned into the internal space (S2) may maintain a suitable condition for cell culture or survival through the dissolution of gas supplied from the gas supply unit 140, 140′.

That is, the solution returned from the accommodating space (S1) of the reactor unit 110 to the internal space (S2) of the solution supply unit 120 by the operation of the circulation pump 130 may be changed into a condition suitable for cell culture or survival through the dissolution of gas supplied from the gas supply unit 140, 140′, and then resupplied to the reactor unit 110.

Accordingly, even when the solution repeatedly circulates through the reactor unit 110 and the solution supply unit 120 via the circulation pump 130, the cells attached to the scaffolds 116 and 116′ may continuously receive the solution in a condition suitable for culture or survival, so that the cells attached to the scaffolds 116 and 116′ may be smoothly cultured or proliferated and may smoothly secrete exosomes.

At this time, the gas supplied to the solution through the gas supply unit 140, 140′ in the internal space (S2) may be in a state where air bubbles are removed, and even when gas is supplied to the solution through the gas supply unit 140, 140′, the generation of air bubbles during the gas supply process may be prevented.

In addition, in the bioreactor system 100 according to an exemplary embodiment of the present invention, the gas supply unit 140, 140′ may be disposed in the internal space (S2) so that the gas can be more smoothly dissolved in the solution.

That is, a main body 141 of the gas supply unit 140, 140′, which will be described later, may be disposed so as to be submerged in the solution filled in the internal space (S2).

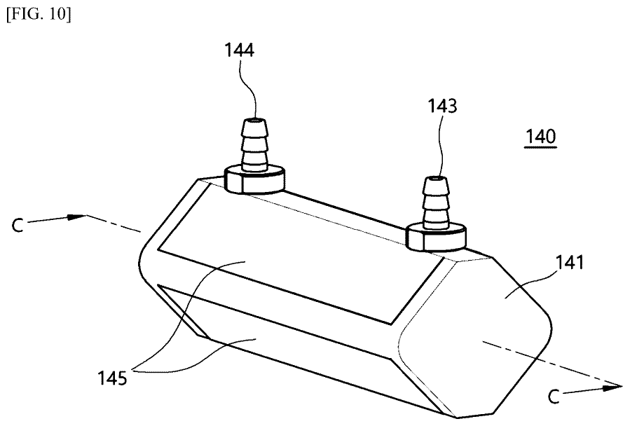

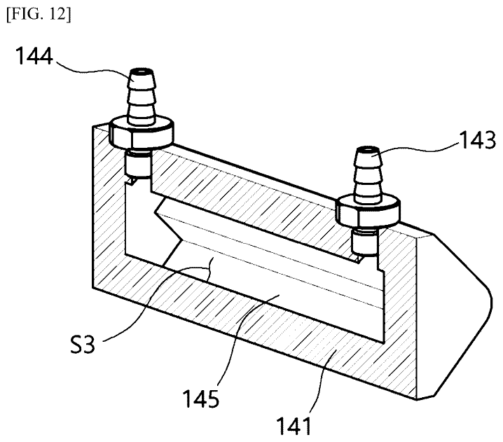

To this end, as shown in FIGS. 10 to 12, the gas supply unit 140, 140′ may include a main body 141 having a gas storage space (S3) filled with gas supplied from the outside, an opening 142 formed in the main body 141 to be in communication with the gas storage space (S3), and a plate-shaped porous member 145 surrounding the main body 141 to cover the opening 142.

In addition, the gas supply unit 140, 140′ may further include a gas inlet 143 provided in the main body 141 to allow gas to flow into the gas storage space S3, and a gas outlet 144 provided in the main body 141 to allow the gas in the gas storage space S3 to be discharged to the outside.

In this case, the gas inlet 143 of the gas supply unit 140, 140′ may be connected to an external gas supply source (not shown), and the gas supply source may supply a suitable type of gas for cell culture or survival, as described above, to the gas supply unit 140, 140′.

Here, the gas supplied from the gas supply source may be carbon dioxide, but is not limited thereto and may be appropriately changed depending on the type of cells, and the gas supplied from the gas supply source may be a mixed gas containing carbon dioxide, oxygen, and the like. In addition, when the gas supplied from the gas supply source includes oxygen, the gas may be a mixed gas having an oxygen concentration of 2 to 14%.

Accordingly, the gas supplied from the gas supply source may be introduced into the gas storage space (S3) of the main body 141, and the gas introduced into the gas storage space (S3) may pass through the porous member 145 surrounding the main body 141 via the opening 142 and then move toward the solution filled in the internal space (S2).

Through this, the bioreactor system 100 according to an exemplary embodiment of the present invention can easily adjust the cell culture environment or survival environment by allowing the type and concentration of gas supplied from the gas supply source to the gas supply unit 140, 140′ to be easily changed, even when the internal space (S2) is implemented as a sealed space.

In this case, the opening 142 formed in the main body 141 may be provided to have a predetermined area, and the porous member 145 may be attached to the main body 141 so as to completely cover the opening 142.

In addition, the main body 141 may be disposed to be submerged in the solution filled in the internal space (S2), as described above.

Accordingly, the gas stored in the gas storage space (S3) may have air bubbles removed while passing through the porous member 145 covering the opening 142, and may be dispersed while passing through the porous member 145 having a predetermined area, thereby moving toward the solution over a wide area.

That is, the gas stored in the gas storage space (S3) may move toward the solution through a wide area corresponding to the surface area of the porous member 145, which directly contacts the solution while corresponding to the area of the opening 142 in the internal space (S2).

Through this, the bioreactor system 100 according to an exemplary embodiment of the present invention may allow the gas supplied from the gas supply unit 140, 140′ to be more smoothly dissolved in the solution filled in the internal space (S2), and may prevent the generation of air bubbles during the gas supply process, even when the gas is supplied from the gas supply unit 140, 140′ toward the solution.

In this case, the gas inlet 143 of the gas supply unit 140, 140′ may serve as an inlet through which the gas supplied from the gas supply source is introduced into the gas storage space (S3), and the gas outlet 144 may serve to regulate the internal pressure of the gas storage space (S3) by allowing the gas present in the gas storage space (S3) to be discharged to the outside during the process of supplying gas into the gas storage space (S3).

Accordingly, even when gas is supplied from the gas supply source into the gas storage space (S3) through the gas inlet 143, the internal pressure of the gas storage space (S3) may be regulated through the gas outlet 144, thereby preventing the generation of air bubbles that may occur due to excessive pressure as the gas stored in the gas storage space (S3) passes through the porous member 145.

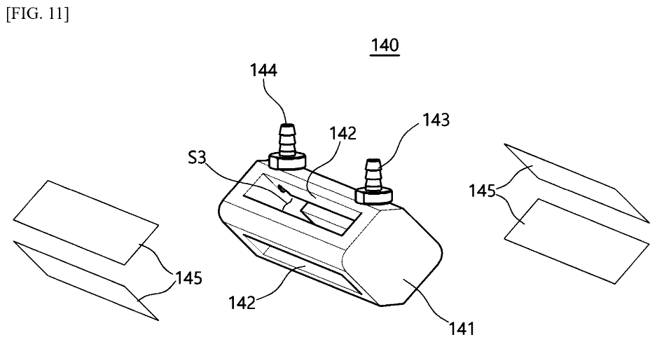

As a specific example, the main body 141 may be formed in the hollow shape of an octahedron with a substantially hexagonal cross-section, as shown in FIGS. 10 and 11, and the gas storage space (S3) may be an internal space of the main body 141.

In addition, the opening 142 may be formed to penetrate, each with a predetermined area, four surfaces that form side surfaces among the eight surfaces of the main body 141, and the gas inlet 143 and the gas outlet 144 may be provided on one surface of the main body 141, which forms an upper surface among the eight surfaces of the main body 141, so as to be in communication with the gas storage space (S3).

In this case, the gas inlet 143 and the gas outlet 144 may be directly fastened to the supply housing 121 with a portion of their lengths protruding outward from the supply housing 121, but the gas inlet 143 and the gas outlet 144 may also be fastened to the supply housing 121 via separate connection pipes 153a and 153b.

As a non-limiting example, as shown in FIGS. 7 to 9, the supply housing 121 may include two fittings 128a and 128b provided on the lid member 123, and the gas inlet 143 and the gas outlet 144 may be respectively fastened to the fittings 128a and 128b via separate connection pipes 153a and 153b.

Through this, the gas supply unit 140, 140′ may be disposed in the internal space (S2) in a state spaced apart from the lid member 123 by a predetermined distance via the connection pipes 153a and 153b, and the main body 141 may be disposed to be submerged in the solution filled in the internal space (S2).

In the drawings, the opening 142 and the porous member 145 are illustrated as being provided in sets of four, respectively, but the present invention is not limited thereto, and the total number of the openings 142 and the porous members 145 may be appropriately changed according to design conditions.

In addition, the main body 141 may also be modified into various known shapes, as long as it has an internal gas storage space (S3) and includes an opening 142 formed to have a predetermined area so as to be in communication with the gas storage space (S3) formed therein.

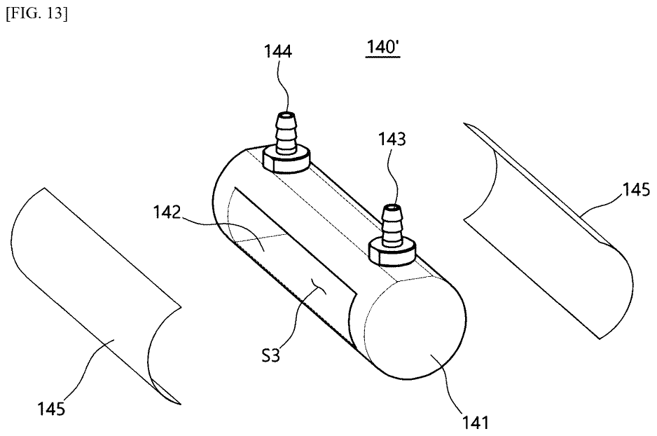

For example, as shown in FIG. 13, in the gas supply unit 140′, the main body 141 may be formed in a substantially hollow cylindrical shape, and the gas storage space (S3) may be an internal space of the main body 141. In addition, the main body 141 may include two openings 142 formed to penetrate a portion of the entire circumferential surface, and the two porous members 145 may be attached to the main body 141 to respectively cover the two openings 142.

In addition, in the drawings, the openings 142 and the porous members 145 are illustrated as being in a one-to-one correspondence, but the present invention is not limited thereto. When a plurality of openings 142 are provided, the plurality of openings may all be covered by a single porous member, and the total number of the openings 142 and the total number of the porous members 145 may be appropriately changed.

In this case, the porous member 145 covering the opening 142 in the gas supply unit 140, 140′ may be provided to allow the gas to move from the gas storage space (S3) toward the solution through the opening 142, while blocking the solution in the internal space (S2) from moving into the gas storage space (S3).

That is, the porous member 145 may block the passage of foreign substances and liquids, while allowing the passage of gases such as carbon dioxide.

For example, the porous member 145 may be a water-repellent treated nanofiber membrane. However, the material of the porous member 145 is not limited thereto, and any material may be used without limitation as long as it blocks the passage of solid and liquid fluids while allowing the passage of gaseous fluids.

Accordingly, even when gas is supplied from the outside toward the solution filled in the internal space (S2) through the gas supply unit 140, 140′, the solution filled in the internal space (S2) may not be contaminated by other foreign substances.

In addition, since the solution filled in the internal space (S2) may be prevented from moving toward the gas storage space (S3) of the gas supply unit 140, 140′ through the porous member 145, the direction of movement of the gas supplied from the gas supply source to the gas storage space (S3) may always be limited to the direction from the gas storage space (S3) toward the solution surrounding the outside of the main body 141.

Accordingly, in the bioreactor system 100 according to an exemplary embodiment of the present invention, since the flow direction of the gas between the gas storage space (S3) and the solution can be consistently maintained, it is possible to fundamentally prevent the generation of air bubbles even during the movement of the gas through the porous member 145 from the gas storage space (S3).

Meanwhile, as described above, when the internal space (S2) is partitioned into a first space (S21) and a second space (S22) by a partition member 126, the main body 141 may be disposed to be located in the first space (S21), and may be arranged to be submerged in the solution filled in the first space (S21).

That is, the main body 141 may be disposed at a position close to the return port 124, which returns the solution from the reactor unit 110 in the solution supply unit 120.

Accordingly, the solution returned from the accommodating space (S1) of the reactor unit 110 to the first space (S21) through the return port 124 may receive gas from the main body 141 disposed in the first space (S21), and then be resupplied to the accommodating space (S1) of the reactor unit 110 through the discharge port 125.

Through this, the gas supplied from the gas supply unit 140, 140′ can be provided with sufficient time to be dissolved in the solution.

Accordingly, the solution supplied to the accommodating space (S1) of the reactor unit 110 through the discharge port 125 may move to the accommodating space (S1) after being changed into a state in which the gas supplied from the gas supply unit 140, 140′ is sufficiently dissolved.

Meanwhile, the bioreactor system 100 according to an exemplary embodiment of the present invention may further include a sterilization filter 160 connected to the gas inlet 143 in the gas supply unit 140, 140′.

For example, as shown in FIG. 7, the sterilization filter 160 may be provided to be connected to the fittings 128a and 128b provided in the supply housing 121.

Accordingly, the gas supplied from the gas supply source may pass through the sterilization filter 160 and then be supplied to the gas supply unit 140, 140′.

As a result, sterilized gas may always be introduced into the gas storage space (S3), thereby preventing contamination of the solution filled in the internal space (S2) by bacteria or microorganisms in advance.

For example, the sterilization filter 160 may be a known sterilizing syringe filter, but is not limited thereto, and various known sterilization filters may be employed as long as they can supply the gas from the gas supply source to the gas supply unit 140, 140′ in a sterilized state.

Meanwhile, the bioreactor system 100 according to an exemplary embodiment of the present invention may further include a frame unit 150 for fixing or mounting the reactor unit 110, the solution supply unit 120, and the circulation pump 130.

That is, the bioreactor system 100 according to an exemplary embodiment of the present invention may be configured in the form of a single module by fixing or mounting the reactor unit 110, the solution supply unit 120, and the circulation pump 130 to one side of the frame unit 150.

Accordingly, the bioreactor system 100 according to an exemplary embodiment of the present invention may be implemented in a portable form, allowing cells to be cultured or proliferated and exosomes secreted from the cells to be obtained in various locations without spatial limitations.

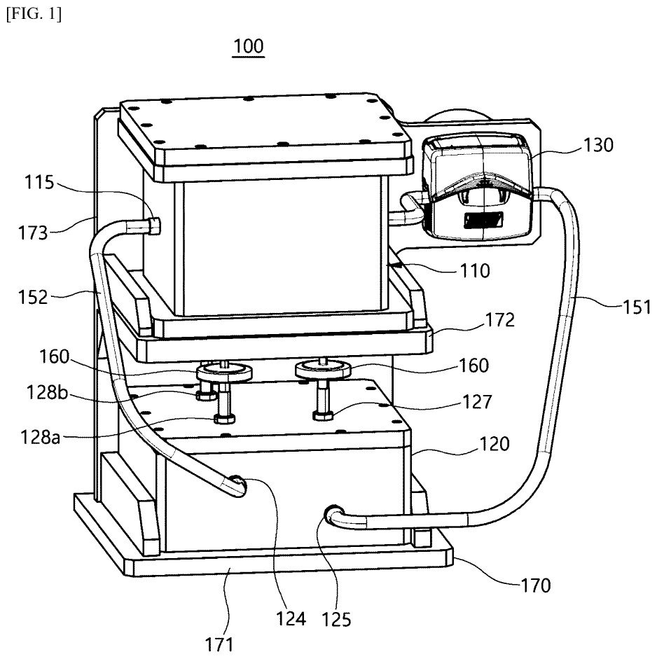

To this end, the frame unit 150 may include a first mounting base 171, a second mounting base 172, and a support plate 173, as shown in FIG. 1.

For example, the first mounting base 171, the second mounting base 172, and the support plate 173 may each be a plate-shaped member having a predetermined area, the second mounting base 172 may be disposed to be positioned above the first mounting base 171, and the support plate 173 may connect the first mounting base 171 and the second mounting base 172.

Accordingly, the first mounting base 171 and the second mounting base 172 may remain spaced apart from each other by the support plate 173.

In this case, the solution supply unit 120 may be disposed on one surface of the first mounting base 171, the reactor unit 110 may be disposed on one surface of the second mounting base 172, and the circulation pump 130 may be mounted on the support plate 173.

Accordingly, in the bioreactor system 100 according to an exemplary embodiment of the present invention, the reactor unit 110, the solution supply unit 120, and the circulation pump 130 may be respectively mounted on the frame unit 150.

As a result, the bioreactor system 100 according to an exemplary embodiment of the present invention may be implemented in a portable form, as described above.

Through this, the user may conveniently carry or transport the bioreactor system 100 according to an exemplary embodiment of the present invention, and thus may transport the bioreactor system 100 to various locations without being restricted by place.

Here, the various locations may be places that provide an environment in which the cells attached to the scaffolds 116 and 116′ can be stably cultured or proliferated, or in which exosomes can be smoothly secreted from the cells. For example, the various locations may be small-scale incubators provided in a laboratory or research facility, and the small-scale incubators may be spaces in which the temperature is maintained at a constant level.

As such, the bioreactor system 100 according to an exemplary embodiment of the present invention may be implemented as a closed circulation system in which the reactor unit 110, the circulation pump 130, and the solution supply unit 120 are connected to each other via connection lines 151 and 152, and the medium stored in the solution supply unit 120 circulates through the solution supply unit 120 and the reactor unit 110 via the circulation pump 130. In addition, even when the solution circulated through the circulation pump 130 is reused, the bioreactor system 100 according to an exemplary embodiment of the present invention can maintain the solution in a suitable state for cell culture or survival through the dissolution of gas supplied from the gas supply unit 140, 140′, thereby minimizing the amount of solution used and thus reducing production costs.

In addition, the bioreactor system 100 according to an exemplary embodiment of the present invention may allow the supply amount, supply cycle, and supply time of the gas supplied from the gas supply unit 140, 140′ to the solution supply unit 120 to be controlled via a separate controller (not shown), and the controller may also control the overall operation in conjunction with the driving of the circulation pump 130.

Also, in the bioreactor system 100 according to an exemplary embodiment of the present invention, gas from which air bubbles have been removed can be supplied to the solution through the gas supply units 140 and 140′, thereby not only enabling stable cell culture but also increasing the amount of exosome secretion.

In addition, the bioreactor system 100 according to an exemplary embodiment of the present invention allows the oxygen concentration of the gas supplied through the gas supply units 140 and 140′ to be easily adjusted, thereby enabling cell culture or exosome production in an environment similar to that of the human body.

In addition, the bioreactor system 100 according to an exemplary embodiment of the present invention can be implemented in a portable form, allowing cells to be cultured or proliferated and exosomes secreted from the cells to be obtained in various locations without spatial limitations.

Meanwhile, in the above description, the cells to be cultured or proliferated or to secrete exosomes in the reactor unit 110 have been described as adherent cells. However, the present invention is not limited thereto, and the cells to be cultured or proliferated or to secrete exosomes in the reactor unit 110 may also be suspension cells. In such a case, the suspension cells may receive signaling and undergo culture/proliferation or secrete exosomes while repeating a contact-suspension-contact-suspension process, in which the cells do not adhere to the scaffold but intermittently attach to and detach from the surface of the scaffold while floating in the solution.

Although exemplary embodiments of the present invention have been described above, the scope of the present invention is not limited to the embodiments set forth herein. Those skilled in the art who understand the spirit of the present invention may readily propose other embodiments by adding, modifying, deleting, or supplementing components within the scope of the present invention, and such embodiments should also be regarded as falling within the scope of the present invention.

What is claimed is:

1. A bioreactor system, comprising:
a reactor unit for culturing or proliferating cells or for secretion of exosomes from the cells;
a solution supply unit, wherein a predetermined amount of a solution to be supplied to the reactor unit is stored in the solution supply unit;
a circulation pump, wherein the circulation pump is connected to the reactor unit and the solution supply unit via a tube and configured to circulate the solution stored in the solution supply unit; and
a gas supply unit, wherein the gas supply unit comprises a main body having a gas storage space filled with a gas supplied from outside, and the gas supply unit is installed in the solution supply unit to supply the gas stored in the gas storage space to the solution stored in the solution supply unit,
wherein the main body is arranged to be submerged in the solution filled in the solution supply unit so that the gas stored in the gas storage space can toward the solution and dissolve.

2. The bioreactor system of claim 1, wherein the gas supply unit comprises:
the main body having the gas storage space filled with the gas supplied from the outside;
an opening formed in the main body to communicate with the gas storage space;
a plate-shaped porous member surrounding the main body to cover the opening;
a gas inlet provided in the main body to allow the gas to flow into the gas storage space; and
a gas outlet provided in the main body to allow the gas in the gas storage space to be discharged to the outside.

3. The bioreactor system of claim 2, wherein the plate-shaped porous member is formed of a hydrophobic membrane to allow the gas to move from the gas storage space toward the solution through the opening while preventing the solution stored in the solution supply unit from entering the gas storage space.

4. The bioreactor system of claim 2, further comprising a sterilization filter connected to the gas inlet.

5. The bioreactor system of claim 2, wherein the gas supply unit comprises a plurality of openings spaced apart along a circumferential direction of the main body, and the plurality of openings are formed in the main body to have a predetermined area.

6. The bioreactor system of claim 1, further comprising a frame unit, wherein the frame unit comprises:
a first mounting base for supporting the solution supply unit;
a second mounting base disposed to be spaced apart from the first mounting base by a predetermined distance and configured to support the reactor unit; and
a support plate interconnecting the first mounting base and the second mounting base, wherein the circulation pump is mounted on the support plate.

7. The bioreactor system of claim 1, wherein the reactor unit comprises:
a reactor housing formed in a shape of an enclosure and having an accommodating space;
a plurality of scaffolds provided in a plate shape having a predetermined area and arranged in parallel with each other at a distance, with one surface of each facing each other in the accommodating space;
an inlet provided in the reactor housing to introduce the solution circulated by the circulation pump into the accommodating space; and
an outlet provided in the reactor housing to discharge the solution in the accommodating space to the solution supply unit.

8. The bioreactor system of claim 1, wherein the reactor unit comprises:
a plurality of scaffolds arranged in parallel with each other at a distance, with one surface of each facing each other,
wherein each of the plurality of scaffolds comprises:
a plate-shaped nanofiber membrane coated with a protein motif; and
a support member attached to one surface of the plate-shaped nanofiber membrane via an adhesive layer so as to support the plate-shaped nanofiber membrane.

9. The bioreactor system of claim 1, wherein the solution supply unit comprises:
an enclosure-shaped supply housing having an internal space, wherein the predetermined amount of the solution is stored in the internal space;
a discharge port provided in the enclosure-shaped supply housing to supply the solution stored in the internal space to the reactor unit; and
a return port provided in the enclosure-shaped supply housing to return the solution from the reactor unit to the internal space, and
wherein the main body is disposed to be submerged in the solution filled in the internal space.

10. The bioreactor system of claim 9, wherein the internal space comprises a first space and a second space, wherein the first space and the second space are partitioned from each other while being in communication with each other via a partition member extending a predetermined height from a floor surface of the enclosure-shaped supply housing,
wherein the return port is provided in the enclosure-shaped supply housing to be in communication with the first space, and the discharge port is provided in the enclosure-shaped supply housing to be in communication with the second space, and
wherein the main body is disposed to be submerged in the solution filled in the first space.

11. The bioreactor system of claim 1, wherein the gas is a mixed gas having an oxygen concentration of 2% to 14%.

12. The bioreactor system of claim 1, wherein the solution is a medium or a buffer solution.
